(19) United States
(12) Patent Application Publication
(10) Pub. No.: US 2011/0224655 A1
(43) Pub. Date: Sep. 15, 2011

(54) CENTRAL CORE MULTIFUNCTIONAL CARDIAC DEVICES

(76) Inventors: Samuel J. Asirvatham, Rochester, MN (US); Charles J. Bruce, Rochester, MN (US); Paul A. Friedman, Rochester, MN (US); Giovanni Speziali, Pittsburgh, PA (US)

(21) Appl. No.: 13/063,374

(22) PCT Filed: Sep. 11, 2009
(86) PCT No.: PCT/US09/56690

§ 371 Date: May 31, 2011

Related U.S. Application Data
(60) Provisional application No. 61/096,126, filed on Sep. 11, 2008.

(51) Int. Cl. A61B 17/00

(52) U.S. Cl. 606/1

(57) ABSTRACT

Intracardiac devices that can provide a plurality of functions (e.g., pacing, defibrillation, cardiac assist, or valve replacement) via a single support member and control means.

### CROSS-REFERENCE TO RELATED APPLICATION

This application claims benefit of priority from U.S. Provisional Application Ser. No. 61/096,126, filed on Sep. 11, 2008.

### TECHNICAL FIELD

This document relates to intracardiac devices that can provide a plurality of functions (e.g., pacing, defibrillation, cardiac assist, or valve replacement) via a single support member and control means, or via several support members and a control means.

### BACKGROUND

Implantable cardiac devices can be used to treat a variety of heart problems, including cardiac arrhythmias and conditions such as heart failure, congenital heart disease and sudden cardiac arrest. For example, pacemakers can be used to deliver small amounts of electrical energy to treat abnormally slow heartbeats (bradycardia) by “pacing” the heart to beat at a normal rate. Implantable cardioverter defibrillator (ICD) devices, also called defibrillators, can deliver electrical energy to the heart to treat heart rhythms that lead to sudden cardiac death. For example, ICDs can stop abnormally fast heartbeats (arrhythmias) by delivering low amounts of energy to the heart, and can stop dangerously fast arrhythmias by delivering a high-energy shock (defibrillation) to the heart. All ICDs include pacemakers, and sometimes are referred to as “combination” pacemaker and defibrillators. In addition, implantable devices can be used to deliver cardiac resynchronization therapy (CRT), also known as biventricular pacing. A CRT device can treat uncoordinated beating (dyssynchrony) and reduce the risk of sudden cardiac death by sending tiny amounts of electrical energy to the left and right ventricles, causing the ventricles to contract at the same time. CRT devices with built-in defibrillators are known as CRT-D devices, while CRT devices in pacemakers are known as CRT-P devices.

In addition to the above conditions, defective or diseased heart valves can be surgically replaced with prosthetic valves. This is most commonly done to replace mitral or aortic valves. Further, cardiac assist devices, also known as ventricular assist devices, can be used to support a failing heart that cannot be safely and effectively managed with standard medical therapy. Assist devices can be blood pumps that connect to the left ventricle, for example, to help restore normal circulation. Assist devices can be used for short-term purposes (e.g., to allow the heart to recuperate and return to normal, independent function), or for more long-term purposes (e.g., to support severe end-stage heart failure patients who are waiting for a heart transplant).

### SUMMARY

This document features multifunctional devices that, in general, have a plurality of intracardiac devices attached to a single lead or multiple leads that can provide structural support as well as power/logic for function. The devices provided herein thus can provide a single structural means for treating a combination of heart conditions, including those mentioned above, for example.

In one aspect, this document features an apparatus comprising an elongate support member having a first end and a second end, a controlling means positioned at the first end and an anchor means positioned at the second end, and two or more intracardiac devices positioned on or in the elongate support member between the first end and the second end, wherein the anchor means is adapted for attachment to the myocardium, and wherein the controlling means is adapted to control the action of the two or more intracardiac devices.

The two or more intracardiac devices can be selected from the group consisting of valves, implantable cardioverter defibrillator (ICD) coils, balloons, micro pumps, and piezoelectric elements.

The two or more intracardiac devices can comprise a first balloon and a second balloon, wherein the first balloon is adapted for placement at about the location of a cardiac valve (e.g., a mitral valve), and wherein the second balloon is adapted for placement in the ventricle distal to the cardiac valve.

The two or more intracardiac devices can comprise a valve and an ICD coil. The ICD coil can be distal to the valve. The apparatus can further comprise one or more electrodes on the circumference of the valve.

The two or more intracardiac devices can comprise a valve and a balloon. The balloon can be distal to the valve and can be adapted for placement within a ventricle.

The two or more cardiac devices can comprise a mesh-like, conical-shaped valve. The mesh-like, conical-shaped valve can comprises one or more fabrics, polymers, pericardial tissue, fascial material, or a biological material coated with an anticoagulant. The mesh-like, conical-shaped valve can define one or more openings adapted to permit unidirectional blood flow through the valve.

The apparatus can comprise two or more elongate support members, a stent, and a valve, wherein the two or more elongate support members are attached to the outer surface of the stent.

The elongate support member can be a hollow tube having a lumen extending between the first end and the second end, wherein the apparatus comprises a micro pump contained within the hollow tube, and wherein the hollow tube comprises a wall that defines one or more lateral openings adjacent to the micro pump such that the lumen is in fluid communication with the external surroundings of the elongate support member in the vicinity of the micro pump.

The two or more intracardiac devices can comprise two or more piezoelectric elements. Each of the two or more piezoelectric elements can comprise a radial array. The two or more piezoelectric elements can comprise at least one radial array and at least one linear array. At least one of the piezoelectric elements can be a radial array adapted for placement in the left atrium, at least one of the piezoelectric elements can be a radial array adapted for placement at about the site of the mitral valve, and at least one of the piezoelectric elements can be a radial array adapted for placement in the left ventricle. At least one of the piezoelectric elements can be a radial array adapted for placement in the right atrium, at least one of the piezoelectric elements can be a radial array adapted for placement at about the site of the tricuspid valve, at least one of the piezoelectric elements can be a radial array adapted for placement in the right ventricle, and at least one of the piezoelectric elements can be a linear array adapted for placement in the right ventricle.

The anchor means of the apparatus can be a screw, a hook, or a barb. The apparatus can be adapted for placement in the coronary sinus.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a general depiction of a multifunctional intracardiac device as provided herein.

FIGS. 2A through 2H depict embodiments of devices configured for use at the site of a valve.

FIGS. 3A and 3B depict embodiments of devices having assist capabilities.

FIG. 4 depicts an embodiment of a device that can be used for annular ring contraction and/or balloon annuloplasty.

FIG. 5 depicts an embodiment of a device having probes that can be configured for placement about a valve annulus.

FIG. 6 shows an embodiment of a device that can be configured for use in balloon valvuloplasty.

FIG. 7 depicts an embodiment of a device having a valve with support members positioned around its circumference.

FIGS. 8A and 8B depict embodiments of devices the can have ultrasound function.

FIGS. 9A and 9B show an embodiment of a device having a collapsible valve.

FIG. 10 depicts an embodiment of a device with a valve having cords connecting the valve leaflets to a support member.

FIG. 11 is a depiction of a device with circumferential support members.

FIG. 12 depicts an embodiment of a device as provided herein placed in a coronary sinus.

Like reference symbols in the various drawings indicate like elements.

### DETAILED DESCRIPTION

The devices provided herein can be used to treat patients having a combination of heart conditions. These patients can in general be divided into at least three broad groups, as follows. A first group includes patients who need either a pacemaker or defibrillator, where additional components may be desired that would preserve and/or restore valve integrity. Patients who need a CRT system also can be included in this group. A second group includes patients who have primarily a valve defect, but who may also benefit from an assist and/or an ICD function, as well as pacing and sensing. A third, large group includes patients who are candidates for left ventricular assist devices (LVAD), and who may also need valve restoration/preservation and/or ICD and pacing/sensing. In some cases, biventricular assist devices can be used instead of LVAD. At least some of these groups (e.g., the first and third groups) also may include patients who might prophylactically receive a device to minimize the chance of tricuspid valve regurgitation that can occur with an indwelling lead.

A key feature of the devices provided herein is a support member for attachment of one or more (e.g., one, two, three, four, or five) cardiac components. The support member can be analogous to a pacing lead, and can have a proximal end that attaches to a controlling means (e.g., a power pack that also may contain logic and/or pumps for drugs or for moving air or fluids), a distal portion configured for placement within the heart, and a distal end having an anchoring means for attachment to the myocardium. The support member may or may not extend to the apex of the chamber(s) in which it is to be placed. In some embodiments, the distal portion of a support member can have one or more arms that branch therefrom. Each arm can have an anchoring means at its distal end, although in some cases only a subset of the arms may have anchoring means. Any suitable type of anchoring means can be used (e.g., screw, hook, barb, etc.). The support member can allow for delivery of power, proper positioning of the attached component(s), stability, and redeployment of cardiac component(s) during the placement process. In addition, the support member(s) can sense QRS for pacing and for assist devices, receive biological data with regard to pressure, blood flow, temperature, pH, and physical properties of the heart, and thus can be used to activate, coordinate, and/or optimize the action of the components attached to the support member.

Another feature provided by some embodiments of a device as provided herein is a circumferential stent/annular ring that can be positioned at the level of a valve, for example. While optional in various iterations, such a stent or ring can be used for, e.g., valve stability, pacing at multiple sites, defibrillation (including intramyocardial defibrillation), sealing perivalvular leaks, shrinking the native annulus, or remote annuloplasties. The ability to pace around the periphery of a stent (native annulus) can facilitate cardiac resynchronization.

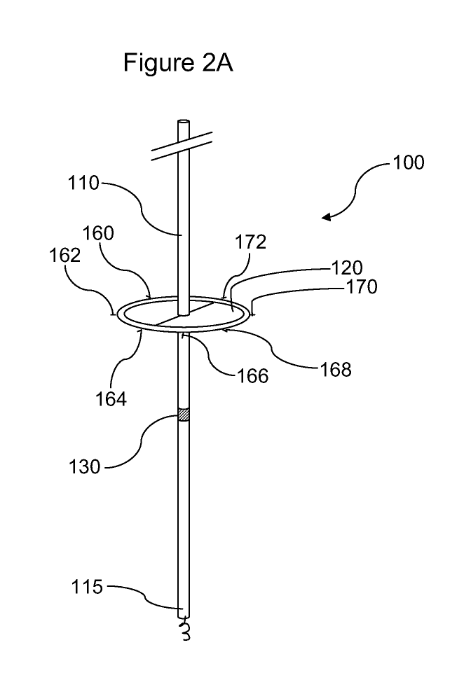

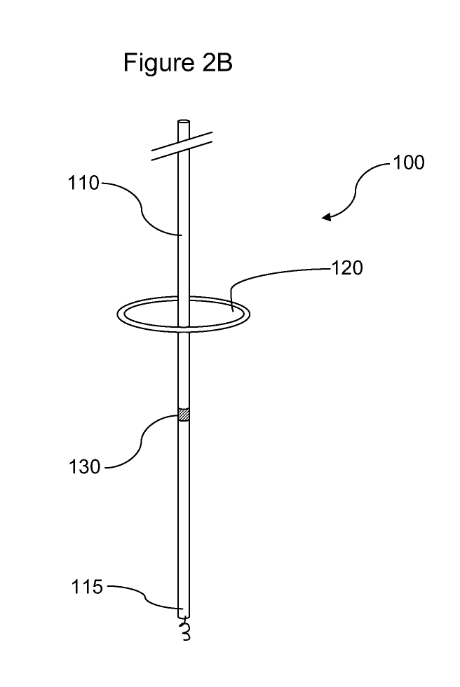

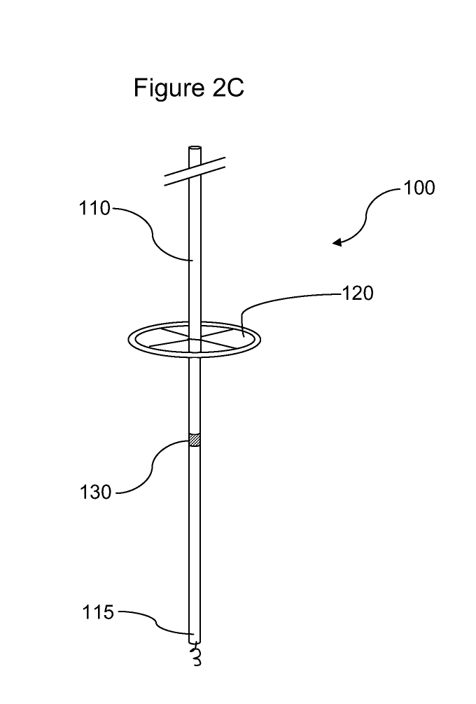

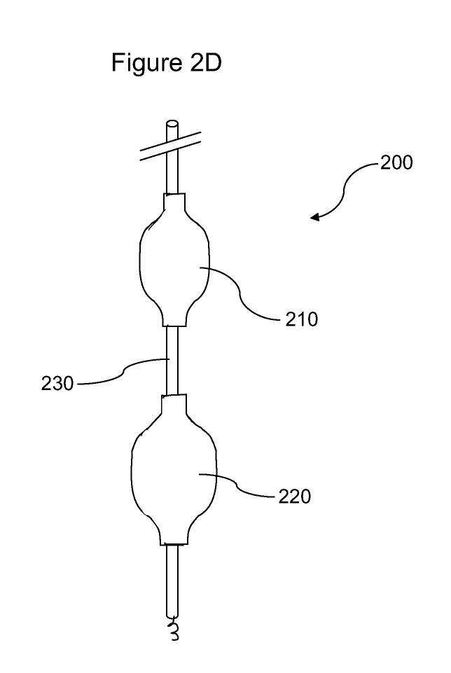

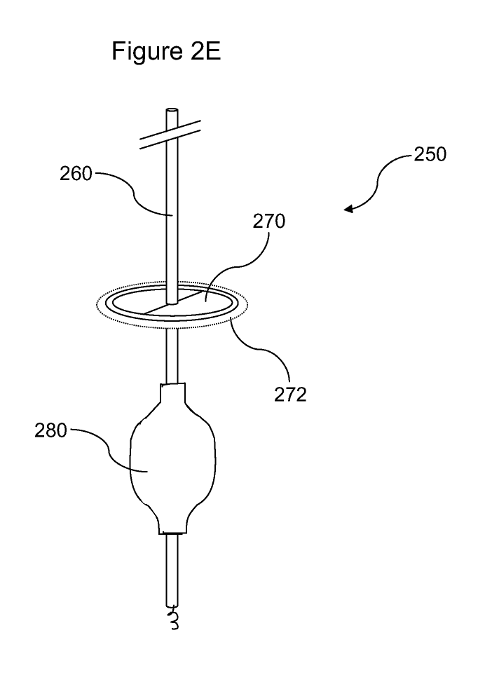

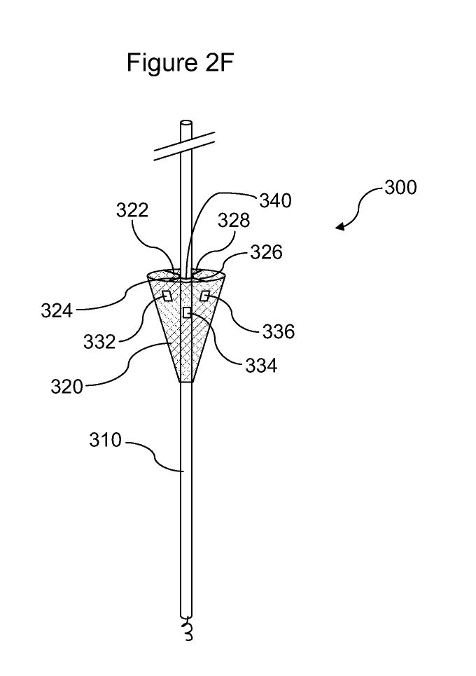

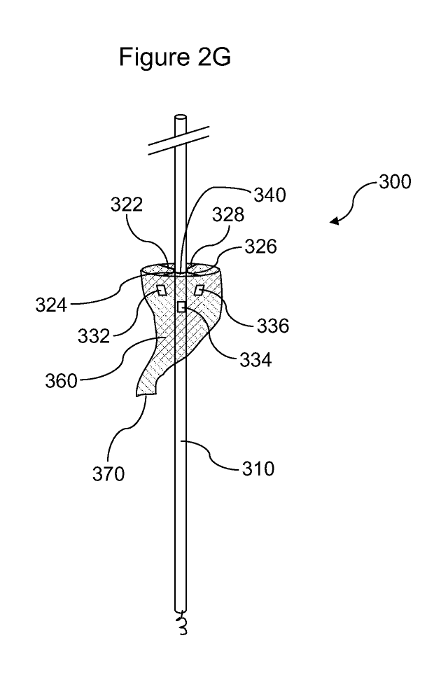

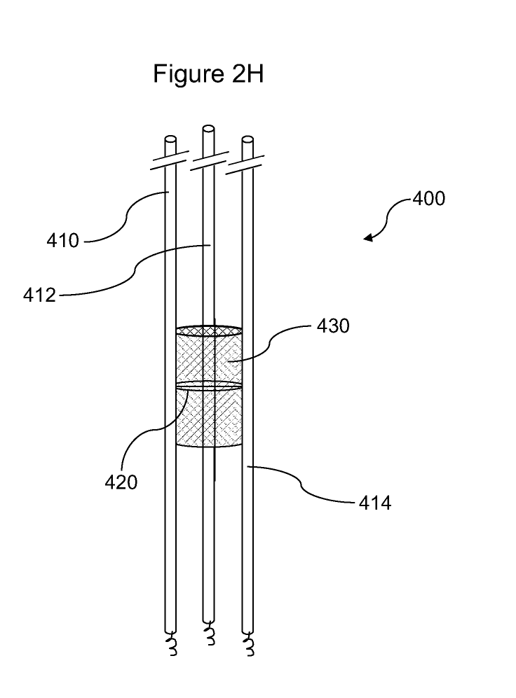

In some embodiments, the devices provided herein do not cross a valve. In such embodiments, for example, an anchoring means can be proximal (i.e., atrial) to the tricuspid valve or the mitral valve. The anchoring mechanism can include one or more struts that can be placed either with a retractable-extendable screw or another suitable mechanism to the atrial myocardium around the tricuspid annulus or the left atrial myocardium around the mitral annulus. Such positioning can be used with, for example, balloon or “windsock” type embodiments such as those depicted in FIGS. 2D, 2F, and 2G and described below. It is noted that embodiments that do not cross a valve would not likely be useful with ventricular pacing, they can have the benefit of completely ameliorating atrio-ventricular valve regurgitation if needed.

A third feature of the devices provided herein, in some embodiments, is the use of QRS timing to coordinate multiple devices and systems for optimal performance, and to independently enhance the performance of multiple systems. For example, in a situation where there is a valve and an assist device, the valve timing and pump function can be syncopated (e.g., with one device having a delay relative to the other) to optimize left ventricular assist device (LVAD) or right ventricular assist device (RVAD) function, as further described below, for example.

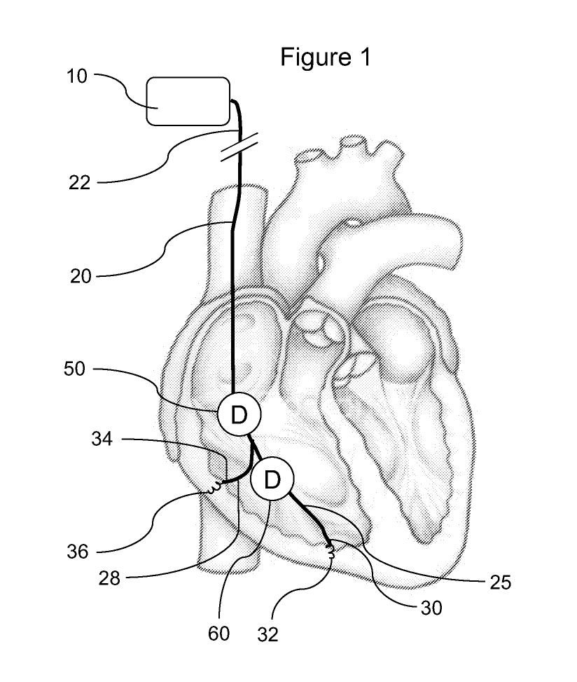

FIG. 1 provides a general depiction of the devices described herein. A controlling means (e.g., a power/logic/fluid delivery device) placed in the body (e.g., in the subclavian/infraclavicular space) can be connected to the proximal end of one or more support members, which can proceed from the controlling means to one or more chambers of the heart. For example, a device as exemplified in FIG. 1 can have controlling means 10 connected to proximal end 22 of support member 20, which can have arms 25 and 28. Arm 25 can have distal end 30 with anchor 32, and arm 28 can have distal end 34 with anchor 36. Anchors 32 and 36 can be embedded in the myocardium, and can provide sensing/diagnostic capabilities (e.g., for sensing QRS, pressure, and/or blood flow information, and/or for providing ultrasound images/piezoelectric elements). In some cases, anchors 32 and 36 can provide therapeutic (e.g., ICD, pacing, and/or anti-coagulum) features.

A support member in any of the embodiments provided herein can be made from any suitable material or combination of materials. For example, a support member can contain silicone, polyurethane, or combinations thereof, can include platinum or titanium type components for electrode and electrode support, and/or may be coated with a dielectric material such as graphite or a similar substance. In some embodiments, different segments of a support member can be made from different materials or can have different compositions, such that the different segments are of varying rigidity. For example, in some cases the proximal portion of a support member may be more rigid than the distal portion, which can be more flexible/pliable. A more flexible/pliable distal end can, for example, prevent the motion of the heart from driving a support member (e.g., an anchor) through the myocardium.

Various intracardiac devices (e.g., intracardiac devices 50 and 60 in FIG. 1) can be mounted on support member 20, and can be positioned either on the same arm of support member 20 or on different arms of support member 20. Intracardiac devices 50 and 60 can include, for example, valves (e.g., tissue and/or mechanical valves), annular rings, balloons, pumps, stents, pacing and/or defibrillation electrodes, radiofrequency (RF) energy delivery electrodes, ICD coils, and combinations thereof. The components of the system can be controlled through the logic of controlling means 10 to allow a device to function in a coordinated manner (e.g., having valve function timed to a pump, pacing timed with a valve, pacing timed to a pump, etc.) via inputs from the sensing components of the system. The support member(s) (e.g., support member 20) can run through the center of the intracardiac device(s) (e.g., intracardiac devices 50 and 60 as shown in FIG. 1), or can run along an outer surface of an intracardiac device (see, e.g., FIG. 2E). Support member 20 can anchor at the apex of a heart chamber or in any other portion of the myocardium, or even in the cardiac sinus. For a device having valve restoration capability, one or more valves (e.g., mitral, aortic, tricuspid, pulmonary, superior vena cava, inferior vena cava, supravalvular aortic, coronary sinus, ventricular or atrial coronary vein, bypass graft, or portal vein valves) can be placed along one or more support members. A valve may be a mechanical valve or a tissue valve, can be adapted for percutaneous delivery via a catheter, and may be mounted on a nitinol or other expandable frame.

It is noted that the devices provided herein can be of variable dimensions (e.g., length and diameter), based on the different intracardiac devices that are incorporated. For example, the length and diameter of the devices provided herein can be similar to those of known cardiac products, such as pacing leads, defibrillator leads, artificial valves, and LVADs.

FIGS. 2A to 2H show exemplary embodiments of various devices configured for use at the site of a valve (e.g., tricuspid valve, mitral valve, aortic valve, or pulmonary valve). For example, FIG. 2A shows device 100, having support member 110 that can provide valve restoration capabilities via valve 120. Distal end 115 of support member 110 can be embedded in the myocardium, and can provide pacing/sensing capabilities with an optional ICD coil (e.g., ICD coil 130) for defibrillation capabilities. Support member 110 can supply power to valve 120 to control its opening and closing, allowing valve 120 to work in conjunction with pacing/defibrillation. Valve 120 can be a traditional mechanical or tissue valve with an opening through which support member 110 can pass. In some embodiments, support member 110 can run along the circumference of valve 120. Valve 120 can have a disc like design, an impeller design, or a rotating “fan” design, as depicted in FIGS. 2A, 2B, and 2C, respectively. Support member 110 can be connected to a power and logic system, which, in cases such as that depicted in FIG. 2B, can run impeller valve 120 to act as a mini-assist device that facilitates blood flow.

In some embodiments, the outer circumference of valve 120 can contain electrodes (e.g., electrodes 160, 162, 164, 166, 168, 170, and 172 in FIG. 2A) that can embed in the myocardium (e.g., via anchors such as coils, hooks, barbs, or pins) to provide pacing, sensing, or ICD function. Electrodes 160, 162, 164, 166, 168, 170, and 172 also can supply RF energy to shrink the valve annulus via fibrosis. In some cases, support member 110 can supply energy to “weld” the annulus tissue to the valve periphery and, in some cases, to activate a mechanical system to perform an annuloplasty (for a related technology see, e.g., U.S. Patent Publication No. 20070135913, which is incorporated herein by reference in its entirety).

FIG. 2D shows device 200, which can have first balloon 210 proximal to second balloon 220, both positioned on support member 230. First balloon 210 can be positioned to span a valve (e.g., the tricuspid valve between the right atrium and the right ventricle or the mitral valve between the left atrium and the left ventricle) to prevent valve regurgitation by filling the space between non-coating leaflets. Second balloon 220 can be positioned within a ventricle, and can inflate and deflate within the ventricle to assist in the pumping of blood. In some embodiments, device 200 can operate as follows. In diastole, both balloons can be in low profile to allow blood to enter the ventricle. During systole, first balloon 210 can inflate to essentially close the valve and prevent regurgitation. After an appropriate delay, second balloon 220 can expand, forcing blood out of the ventricle. Such a system can be deployed in either the right side of the heart or the left side of the heart. Devices such as that shown in FIG. 2D can be made using, for example, methods that are known in the art for making balloon catheters and the like. A balloon can be expanded by purely physical or mechanical forces related to cardiac contractility, or may be assisted with power delivery through a battery source. When mechanical and hemodynamic forces cause deployment of the balloon, it is noted that in cardiac systole the ventricular pressure is higher than the atrial pressure, and thus the portion of the balloon that is placed ventricular to the atrio-ventricular valve can be squeezed or compressed more than the portion of the balloon in the atrium. This can result in asymmetric deformation of a balloon, such that a larger surface area occurs on the atrial aspect serving as the device to close the valve and decrease the amount of tricuspid regurgitation.

FIG. 2E shows device 250, which is analogous to device 200 shown in FIG. 2D, but with a valve at the proximal position rather than a balloon. Thus, device 250 can have support member 260, valve 270, and balloon 280. Rim 272 of valve 270 can have a mechanism (e.g., of fabric, balloon, or polymer) to conform the valve to the patient's annulus anatomy, thereby limiting perivalvular leaks. Distal balloon 280 can act to force blood out of the ventricle in which it is placed (e.g., into the pulmonary artery if balloon 270 is placed in the right ventricle, and into the aorta if balloon 270 is placed in the left ventricle). As for device 200, the valve and balloon components of device 250 can act in concert to optimize valve function with assist function.

FIG. 2F illustrates another embodiment of a device having a valve. Device 300 can include support member 310 and cone-shaped valve 320. The body of cone-shaped valve 320 can be configured to allow blood to flow from the atrium to the ventricle, but to prevent the reverse flow. In some embodiments, device 300 can have annular struts (e.g., struts 322, 324, 326, and 328) between cone-shaped valve 320 and support member 310. The annular struts can be nitinol based or can be hinged with springs, for example, and can provide mechanical support and positioning for cone-shaped valve 320 as well as sites at which pacing and defibrillation can be delivered to the myocardium. In some cases, one or more spikes or needles (e.g., metallic spikes or needles) can extend from an equatorial stent into the myocardium to provide mechanical stability as well as intramyocardial pacing or coils for defibrillation.

The body of cone-shaped valve 320 can contain any suitable material (e.g., any suitable fabric, polymer, or other pliable material, such as DACRON®, materials used to make balloons, bovine pericardial tissue or pericardial tissue from other species such as humans, biologic fascial material such as peritoneum pleura, or other biological material coated with an anticoagulant). In some cases, the material of the valve body can be supported by a nitinol frame (e.g., a compressible fine nitinol mesh covered with either a fabric, nitinol of a different density, or another metal). In addition, cone-shaped valve 320 can have windows, slits, mesh, or other openings (e.g., windows 332, 334, and 336) through the material to allow blood to flow to the ventricle. In addition, an optional sliding ring (e.g., ring 340) along the shaft of support member 310 can keep the system in the correct orientation. During systole, the pressure in the ventricle can cause the material to collapse and seal the valve, preventing regurgitation. During diastole, the valve material can “relax” into its extended shape, allowing blood to flow through the valve. In some cases, as in the embodiment shown in FIG. 2G, device 300 can have cone-shaped valve 360 with off-center outlet 370, which can provide increased sealing of valve 360 during systole.

FIG. 2H depicts device 400, which can have valve 420 mounted on stent 430, with support members 410, 412, and 414 attached to stent 430 to provide support and securement of the system. Support members 410, 412, and 414 also can provide pacing, sensing, or ICD function with the incorporation of ICD coil 440. Device 400 can be made using any suitable method, including techniques that are known in the art for making stents, for example. For example, one, two, three, or more struts can be placed for positioning around the annulus. A valve-like device mounted on a stent can be pre-mounted on the struts. The mesh for the stent portion of the device can be made of nitinol or a similar substance, such that the entire device including the peripheral struts can be compressed into a single sheath. In use, the sheath can be deployed into the right ventricle (or other suitable site) and then pulled back away from the ventricular apex to expose the screw-in mechanism(s) for the annular struts. This can allow for deployment of the struts. By further pulling back the sheath, the valve-like structure mounted on the stent can expand and be deployed at a suitable location on the atrio-ventricular annulus.

Devices having valves as described herein also can be made using any other suitable method, including, for example, those described in U.S. Patent Publication No. 20070093890, PCT Publication No. WO 2007/135101, PCT Publication No. WO 2006/127509, and PCT Publication No. WO 2007/144865, each of which is incorporated herein by reference in its entirety.

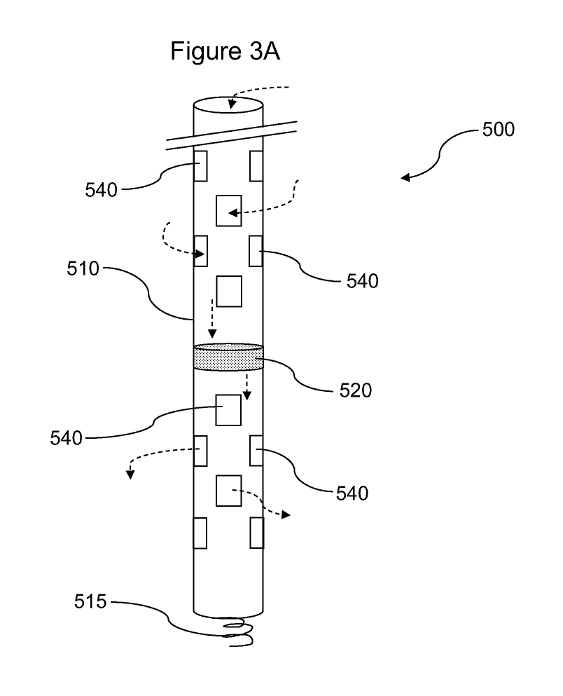

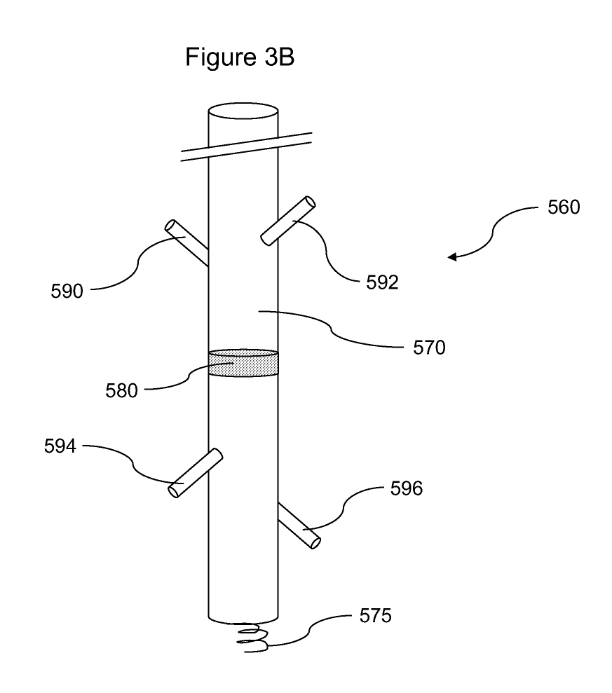

FIG. 3A shows an embodiment of an assist device as provided herein. Device 500 can include hollow support member 510, with micropump 520 positioned within the lumen of support member 510. Micropumps are known in the art, and include, for example, that disclosed in PCT Publication No. WO 2008/034068, which is incorporated herein by reference in its entirety. Device 500 can be powered and controlled by a power/logic system (see, e.g., FIG. 1) and can be combined with pacing/sensing/ICD or valves in a system. When placed into a patient, micropump 520 can be positioned at the site of a valve (e.g., the tricuspid or mitral valve) to assist in the flow of blood between heart chambers. In some embodiments, for example, the device shown in FIG. 3 can assist the flow of blood from the left atrium to the left ventricle (e.g., to treat diastolic heart failure). The orientation of micropump 520 can be determined by the anatomic location at which device 500 will be placed. For example, micropump 520 can be positioned to pump blood toward the distal end of support member 510 (e.g., as indicated by the dashed arrows in FIG. 3A) if device 500 is to be placed through the tricuspid or mitral valve. Alternatively, micropump 520 can be positioned to pump blood away from the distal end of support member 510 if device 500 is to be placed through the aortic or pulmonary valve.

In some cases, device 500 can be timed via a sensing system (e.g., via electrode anchor 515 in the LV myocardium) to activate in conjunction with the patient's natural heart rate. Support member 510 of device 500 can have inlets and/or outlets (e.g., tubes, slits, or windows, such as windows 540 shown in FIG. 3A) proximal and distal to micro pump 520 (i.e., on either side of the native or replacement valve) to allow for passage of blood. In some embodiments, device 500 can include a valve that can replace or augment a natural valve.

FIG. 3B illustrates another embodiment of an assist device. Device 560 can have support member 570, micropump 580, and tubes 590, 592, 594, and 596 to allow blood to flow through device 560. Again, anchor 575 on support member 570 can incorporate sensing and/or pacing electrodes to time endogenous heart contractions with pump activation.

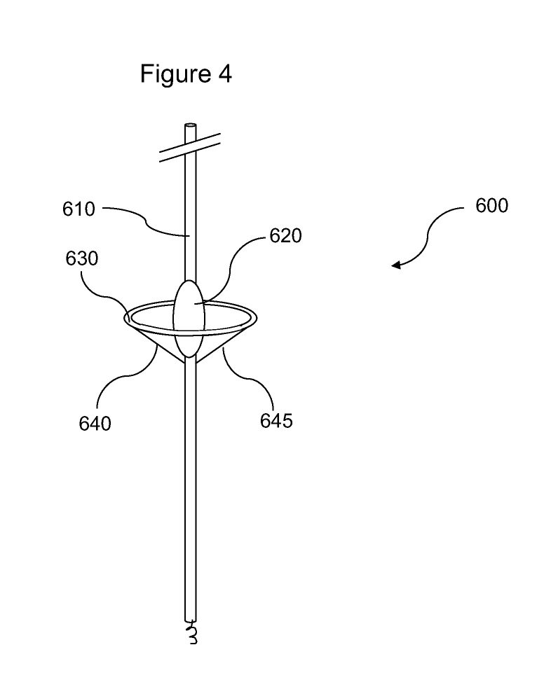

Device 600, shown in FIG. 4, can be used to perform annular ring contractions and/or balloon annuloplasties periodically and in a percutaneous manner. Device 600 can include support member 610, which can have balloon 620, annular ring 630, and optional struts 640 and 645 positioned thereon. Once device 600 is deployed, support member 610 can supply power and/or deliver fluid to activate balloon 620 or to shrink/tighten annular ring 630. Optional struts 640 and 645 can extend between support member 610 and annular ring 630 to supply power and control annular ring 630, for example. In some embodiments, the annular ring control system illustrated in FIG. 4 can be deployed without balloon 620.

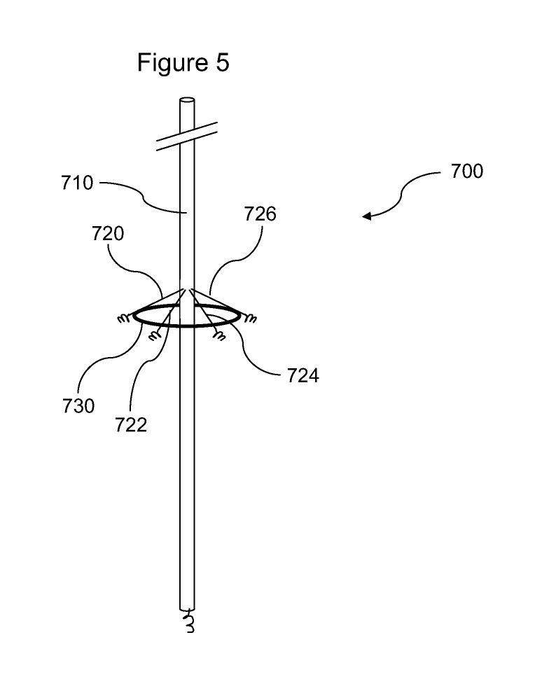

FIG. 5 shows device 700, which can have support member 710 and probes 720, 722, 724, and 726, as well as, in some case, annular ring 730. In some embodiments, the probes can be positioned at a valve annulus and used to deliver RF energy to shrink the annulus. Alternatively or in addition, the probes can provide pacing, sensing, or ICD features.

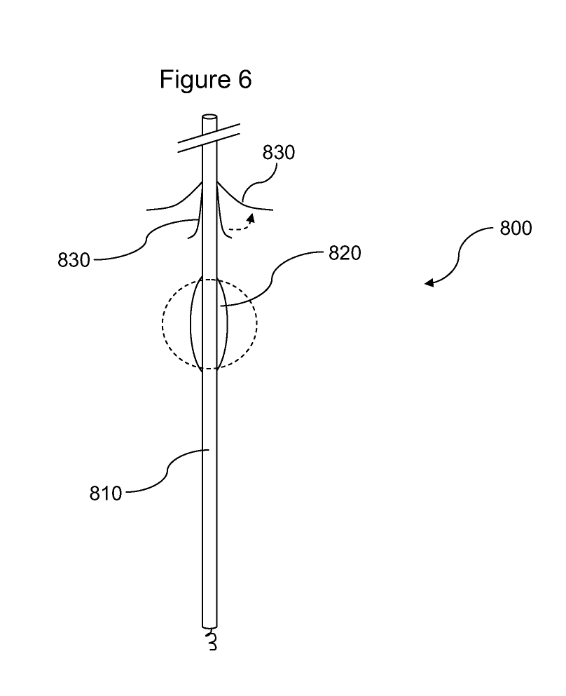

FIG. 6 illustrates a device for percutaneous and repeated balloon valvuloplasties, which can be used to treat valve stenosis. Device 800 can have support member 810 with attached balloon 820. Balloon 820 can be inflated (e.g., as indicated by the dashed oval in FIG. 6) remotely to re-open a stenosed valve. Device 800 optionally can have proximal protection means 830, which can be in any suitable configuration (e.g., a mesh or a cage) that can capture any debris that may be dislodged during the inflation of balloon 820. In some embodiments, protection means 830 can be actuated remotely, such that it can expand (e.g., as indicated by the dashed arrow in FIG. 6) during a valvuloplasty procedure and collapse after the procedure, and can remain collapsed in between procedures.

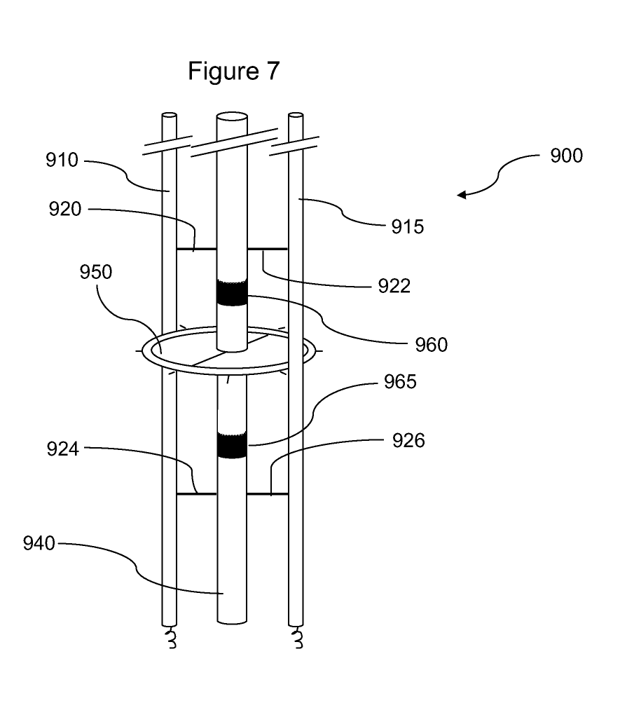

FIG. 7 shows device 900, which is a multi-support system that can be used to deploy, for example, a valve and an assist device. In some embodiments, device 900 can have support members 910 and 915, struts (e.g., struts 920, 922, 924, and 926), with hollow tube 940 mounted between support members 910 and 915 and at least some of the struts. In some cases, device 900 can have more than two support members (e.g., three, four, five, or more than five support members). Device 900 also can have optional valve 950 and one or more micropumps (e.g., micropumps 960 and 965) contained within hollow tube 940. The struts can provide power and control for the pump(s) and the optional valve. In place of traditional valve 950 as shown in FIG. 7, balloons could be inflated above and/or below the native valve to prevent regurgitation, and blood flow could be dependent solely on assist device 900 (see, e.g., U.S. Pat. No. 4,753,221, which is incorporated herein by reference in its entirety). In some embodiments, device 900 can be timed to endogenous heart activity via anchor electrodes attached to the distal ends of support members 910 and 915.

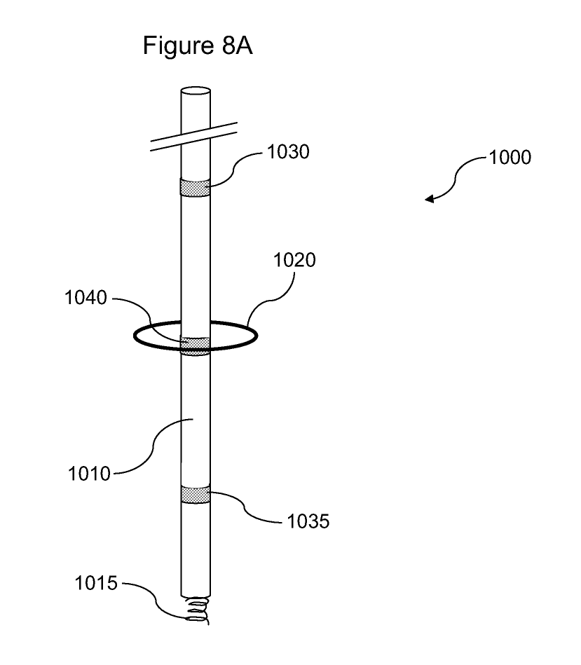

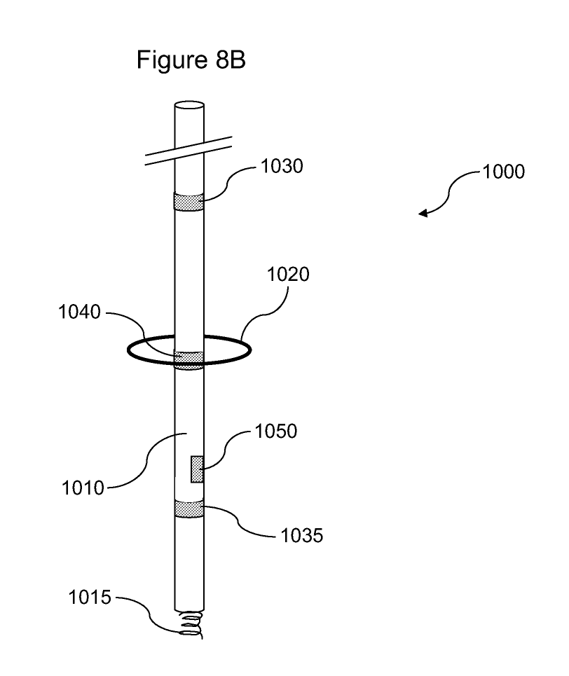

FIGS. 8A and 8B depict a variation that can be incorporated into any of the embodiments described herein—ultrasound imaging. To provide for ultrasound imaging, for example, piezoelectric elements can be incorporated into the support member(s) of any of the devices provided herein to perform one or more of the following exemplary tasks: (1) thrombus detection (e.g., on devices or otherwise); (2) valve function; (3) ventricular function; and (4) therapeutic function. A device can have any suitable number of piezoelectric elements (e.g., one, two, three, four, five, six, seven, eight, nine, ten, or more than ten piezoelectric elements), which can be at any suitable location(s) on or in the device. Ultrasound functions can provide intermittent, periodic imaging for brief periods of time (e.g., less than 1 second, or one full cardiac cycle, or some other specified duration), depending on the indication. In some embodiments, imaging may occur only when a wand is held over a control “can” in the patient's chest. The wand can, for example, contain an inductor that generates a current in the “can” to power the ultrasound and also, in some cases, to recharge the batteries. While such imaging is again intermittent, it can be useful depending on the goal/need. In some embodiments, ultrasound images can be stored in an implanted “can” for later retrieval.

An M-mode ultrasound image can be produced with a single beam in an ultrasound scan, such that movement of a heart valve, for example, can be depicted in a wave-like manner. Because of its high sampling frequency (up to 1000 pulses per second), M-mode ultrasound can be useful in assessing rates and motion, and is routinely used in cardiac imaging.

For phased array ultrasound imaging, an ultrasound transducer can include an array of transducer elements, with a multiple channel transmitter and a multiple channel receiver connected to the transducer. Each transmitter channel can cause a selected transducer array element to transmit an ultrasound pulse into an object being imaged. The transmitted ultrasound energy can be steered and focused by applying appropriate delays to the pulses transmitted from each transducer array element, so that the transmitted energy adds constructively at a desired focal point. The ultrasound energy then can be partially reflected back to the transducer array by various structures and tissues.

In some embodiments, an approach can be taken that is an intermediate between phased array and M-mode. If several M-mode transducers are positioned side by side and a software/computer algorithm is used to “connect the dots,” volumes and 3-D shapes can be generated without all of the hardware requirements of true phased array images. Such an “intelligent” M-mode arrangement may be useful for many applications at significant power consumption savings. Further, it is noted that while traditional phased array necessarily results in a sector image orthogonal to the shaft of the support member, other types of imaging also can be used. These include radial phased array, three-dimensional (3-D) ultrasound, and rotating element ultrasound.

For a radial phased array, instead of having all of the piezoelectric elements in line along the side of the transducer, they can be located around the circumference of the support member at the level of interest (e.g., so they could eventually be positioned in the area of a valve, in an atrium, and/or in a ventricle of a patient). Such positioning can result in a circumferential image with no moving parts. Gaps can be filled algorithmically to reduce piezoelectric element density and to address technical challenges.

3-D ultrasound can be very appealing, particularly given some of the more useful applications. With a modest number of circumferentially positioned ultrasound elements (e.g., a radial phased array) placed at different points along the length of a support member, data from both right ventricle (RV) and left ventricle (LV) points could be collected to permit accurate 3-D volume assessments. This reconstruction can be based solely on the points collected by a central support member ultrasound or, alternatively, real-time data from a central support member can be melded with a 3-D image of the patient's heart obtained via CT or external echo, so that the more limited intracardiac information is algorithmically “expanded.” In some cases, a moving rotating element can perform the function of collecting 3-D information.

Rotating element ultrasound can be achieved in the devices described herein as follows, for example. First, the support member of a device can have a lumen for a stylet. After placement, the stylet can be replaced with a tiny ultrasound probe. For intermittent imaging, or for imaging that is only performed while an external wand provides power, a motor or motion may not be an issue. In some embodiments, a motor or magnet system can be used to move a rotating ICE proximally or distally within the support member, allowing for imaging of the ventricles, atria, and great vessels.

Potential uses for imaging and/or integrated ultrasound in the devices provided herein include, for example, assessment of fluid/volume status, assessment of cardiac output (which can be particularly useful for titration of iterations with balloons to serve as ventricular assist devices), real-time pulmonary and systemic vascular resistance measurements, other hemodynamic assessments (e.g., 3-D RV and LV volumes, RV pressures, and transvalvular pressure gradients, which can be obtained by measuring pressure on the post on both sides of valve). Ultrasound imaging also can be used to assess valvular regurgitation, to detect thrombus formation on a valve, a support member, an atrial appendage, or other structures, for correlation of pressure changes and arrhythmia (e.g., to control therapy, for example, for normotensive arrhythmias treated with ATP rather than shock), and for correlation of valvular regurgitation with pacing site (e.g., to optimize CRT with regard to cardiac output and to minimize valvular regurgitation). Further, ultrasound could prevent thrombus formation. Since stasis is part of Virchow's triad for thrombus formation, periodic application of mechanical vibration (i.e., ultrasound energy) may keep the blood that contacts the support member(s) and the valve structure “agitated” and prevent it from forming clots. If clots are seen, higher energy (i.e., focused) vibration or ultrasound may be useful to dissolve the clots and render them harmless.

For devices that contain a plurality of ultrasound elements, it may be beneficial to limit the number of wires. In some embodiments, a single wire can serve all of the elements, but can have each one at a slightly different frequency so that frequency “keyed” activation of the piezoelectric elements can control which element is activated by a specific pulse.

FIGS. 8A and 8B depict embodiments of intracardiac devices having ultrasound capabilities. Device 1000 can include support member 1010 with anchor 1015, optional valve or annular ring 1020, and piezoelectric elements 1030, 1035, and 1040. In some embodiments, piezoelectric elements 1030 and 1035 can be positioned for placement in different chambers of the heart, with piezoelectric element 1040 positioned for placement at about the location of the valve between the different chambers. For example, piezoelectric element 1030 can be adapted for placement in the left atrium, piezoelectric element 1035 can be adapted for placement in the left ventricle, and piezoelectric element 1040 can be adapted for placement at about the location of the mitral valve. The piezoelectric elements can be, independently, radial array or linear array elements. In some cases, for example, piezoelectric elements 1030, 1035, and 1040 can be radial array elements, such that piezoelectric element 1030 can be used to detect thrombus formation in the left atrium, piezoelectric element 1035 can be used to assess LV function, and piezoelectric element 1040 can be used to assess function of the mitral valve, for example.

In some embodiments, e.g., as depicted in FIG. 8B, a device can include both radial array and linear array piezoelectric elements. For example, device 1000 can include support member 1010 with anchor 1015, optional valve or annular ring 1020, and piezoelectric elements 1030, 1035, 1040, and 1050. In some cases, piezoelectric element 1030 can be adapted for placement in the right atrium, piezoelectric elements 1035 and 1050 can be adapted for placement in the right ventricle, and piezoelectric element 1040 can be adapted for placement at about the location of the tricuspid valve. Again, the piezoelectric elements can be, independently, radial array or linear array elements. In some cases, for example, piezoelectric elements 1030, 1035, and 1040 can be radial array elements, and piezoelectric element 1050 can be a linear array element. In such embodiments, piezoelectric element 1030 can be used to detect thrombus formation in the right atrium, piezoelectric element 1035 can be used to assess RV function, piezoelectric element 1040 can be used to assess function of the tricuspid valve, and piezoelectric element 1050 can be used to assess function of both the RV and the LV, for example.

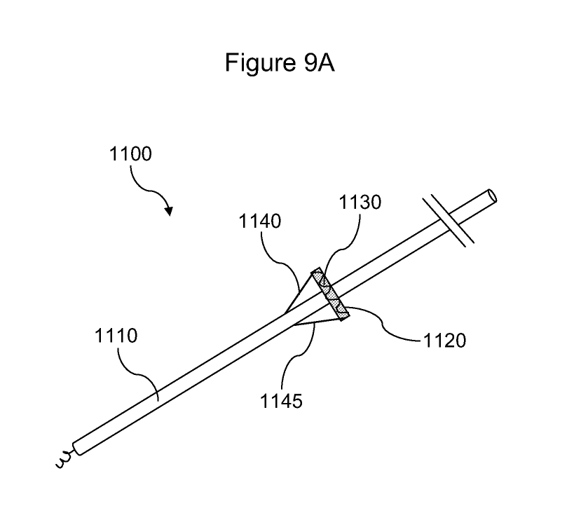

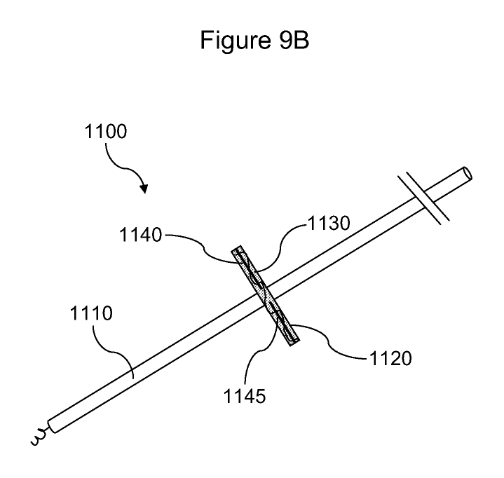

FIGS. 9A and 9B illustrate a method for deploying a valve device as described herein in a percutaneous manner. FIG. 9A shows device 1100 in a collapsed configuration, such that valve 1120 is adjacent to support member 1110 (e.g., as it can be positioned prior to placement). Valve 1120 can be mounted on annular ring 1130, which in some embodiments can have shape memory (e.g., can be made from a material such as nitinol). In some cases, annular ring 1130 can be held in a “closed” configuration by control struts connected to support member 1110 (e.g., struts 1140 and 1145). After placement within the heart of a patient, annular ring 1130 can expand by virtue of its shape memory capability, or can be manually expanded by the control struts. See, e.g., FIG. 9B. Device 1100 can be placed within a patient by, for example, passage through a hollow catheter, and can expand or be expanded upon its exit from the distal end of the catheter.

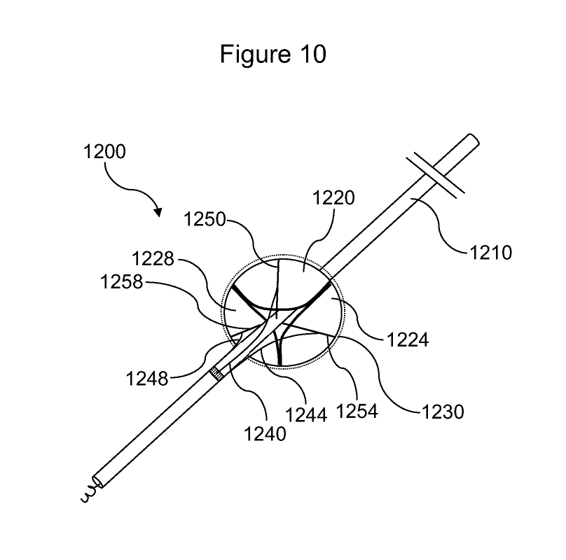

FIG. 10 shows a valve design that can be incorporated into a multi-functional system as described herein. Device 1200 can have support member 1210 and one or more valve leaflets (e.g., valve leaflets 1220, 1224, and 1228), which can be mounted on collapsible/expandable frame 1230. The leaflets can be made of any suitable flexible material (e.g., a fabric such as PTFE, Dacron, or any other suitable material). Collapsible/expandable frame 1230 can be, for example, a ring made from a shape memory substance such as nitinol. The leaflets can be attached to support member 1210 via cords (e.g., cords 1240, 1244, and 1248) to prevent prolapse of the leaflets into the atrium. In addition, device 1200 can have struts (e.g., struts 1250, 1254, and 1258) extending between support member 1210 and frame 1230. The struts can, for example, provide centering and stability to the device, and in some embodiments, can provide power to the circumference of frame 1230 for pacing/sensing/ICD electrodes along the circumference.

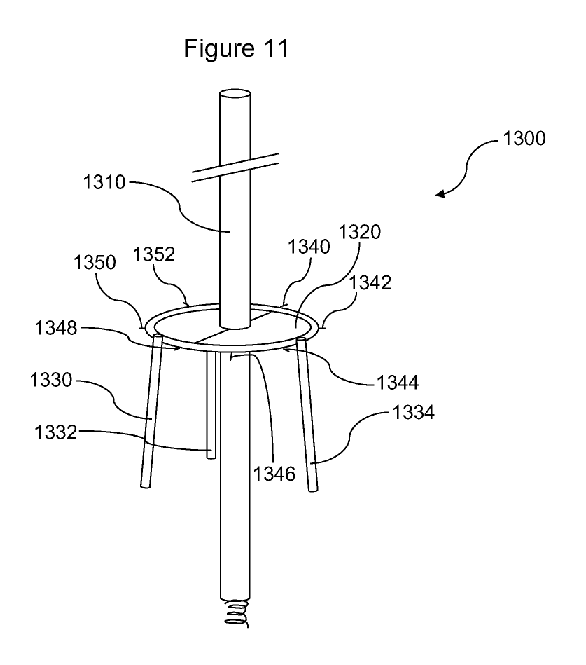

FIG. 11 shows device 1300, which is another example of a device having a valve and pacing/sensing/ICD capability. Device 1300 can include central support member 1310, valve 1320, and one or more circumferential support members (e.g., circumferential support members 1330, 1332, and 1334), which can be positioned to extend along the endocardial surface of the heart. The circumferential support members can provide mechanical support as well as pacing and defibrillation functions. In some embodiments, device 1300 can have one or more intramyocardial spikes (e.g., spikes 1340, 1342, 1344, 1346, 1348, 1350, and 1352) on the circumference of valve 1320, which also can provide pacing and defibrillation functions.

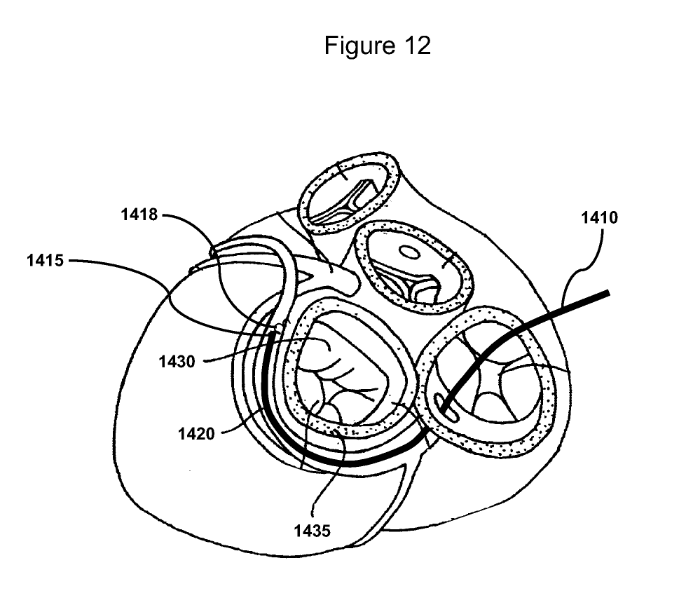

In some embodiments, a support member of a device as provided herein can be placed in the coronary sinus of a patient. As depicted in FIG. 12, for example, support member 1410 can be placed into coronary sinus 1420. Support member 1410 can have distal end 1415 with electrode/anchor 1418, and a proximal end connected to a power source. Electrode/anchor 1418 can supply energy to the heart muscle to sense, pace, and/or defibrillate the heart. In some cases, the power source also can provide energy to the body of support member 1410 where it nears mitral valve 1430, which can be used to shrink mitral valve annulus 1435, or which can be used to adhere support member 1410 to the vessel wall near mitral valve annulus 1435 so that the power supply can then provide mechanical energy to shrink annulus 1435.

The devices described herein can be timed based on, for example, electrocardiogram (ECG) signals. In some embodiments, ECG timing can be used to program a device. For valve function, for example, an atrioventricular valve (either the tricuspid or the mitral valve) will need to open in diastole and be closed during systole. Electrograms from a valve device, received via the support member(s), the valve, or both, can be used to effect this timing. When a ventricular electrogram is sent, systole starts and the atrioventricular valve can be closed for this period of time. Since systole is approximately 4/9 of the cardiac cycle, the interval between two preceding ventricular electrograms can be used to calculate the cycle length of the cardiac activation, and 4/9 of this cycle length can be the time of closure of the electrically assisted percutaneously placed valve. Following this length of time, the valve can be mechanically opened. Further refinement for atrioventricular valves can include the use of sensed atrial electrograms (A wave), such that the valve does not close prior to, during, or for about 100 ms after sensing of the atrial electrograms. This can optimize diastolic filling of the ventricle by preventing premature closure of the valve. Timing cycles relevant for atrioventricular valve closure can be incorporated into existing standard timing cycles for pacemakers, which can be include in some embodiments of the devices described herein.

For function of a semilunar valve (either the aortic valve or the pulmonary valve), active valve opening can be triggered by sensing of the ventricular electrogram (R wave). The duration of valve opening and initiation of active closure can be calculated for the period of systole as explained above for atrioventricular valves.

In cases of ventricular arrhythmia or atrial fibrillation, timing cycles for valve opening or closing may or may not be linked to electrical activation. An algorithm to diagnose ventricular tachycardia above a certain rate (continuous sensing of R-R intervals) can be used to diagnose arrhythmia and, in some embodiments, to create a fall back option for valve function either to a standard rate or continued sensing of ventricular function, or to create a fall back function for a coexisting LVAD.

ECG timing also can be used to program ventricular assist devices (e.g., LVADs). When a percutaneous LVAD is to be used, for example, timing of the device can be linked to both valve opening and valve closing, the pacemaker, and the LVAD itself. In some embodiments, the sequence of events can be as follows. (1) If an intrinsic R wave is sensed, then a semilunar (aortic or pulmonary) valve can open and be kept open for 4/9 of the cardiac cycle. (2) If no R wave is sensed and a pacemaker is present, the pacemaker can attempt to stimulate the ventricle. (3) If ventricular stimulation results in sufficient mechanical contraction to open the valve without electrical assist, then such can be allowed to happen. (4) If ventricular stimulation does not open the semilunar valve, then electrical assist to the valve can be given. (5) If a pressure sensor placed distal to the valve detects low and possibly life-threatening low pressure, despite ventricular stimulation and mechanical opening of the valve, then left ventricular assist through the LVAD can become operative. (6) The device can continue to look for electrograms, and if spontaneous R waves are noted the device can fall back to event (1) above. (7) If, on the other hand, multiple R waves are detected and occur more frequently than a certain cut off rate, the pressure sensor can determine whether the assist device will become operative immediately, or electrical assist or other pacing stimulation can be attempted (e.g., at very low pressure, and then the LVAD can be activated.

In some embodiments of an LVAD (e.g., as depicted in FIGS. 3A, 3B, and 7), the sensing electrodes of the anchors and the power/logic system can be used to pick up ventricular electrograms. In such embodiments, the device can be in place but not functioning all the time in patients who do not have heart failure or critical valvular disease. When arrhythmia, occurs the sensors can send a signal that activates the pump and maintains circulation. The arrhythmia may then terminate by itself, or a controlled cardioversion can be done with the patient sedated. The device also can serve as a backup for patients who have existing defibrillators, should they exhaust ICD therapy. Finally, such devices can incorporate ICD coils to perform traditional defibrillation and activate the pump if defibrillation fails.

The devices described herein can be placed in a patient using methods that are well established in the art. For example, intracardiac placement of devices such as pacemaker leads is routinely carried out. Typically, a small incision can be made in the chest wall, and the leads can be threaded through the incision into a large blood vessel in the upper chest, and then into the heart. Using the same incision, a small pocket can be created under the skin to hold a pulse generator, and the pacemaker leads can be hooked up to the pulse generator.

Similar methods can be used to place the devices described herein within a patient. For example, the support member(s) of a device, and any components (e.g., valves, rings, balloons, piezoelectric elements, or other components) positioned therein or thereon can be positioned within a patient's heart using methods analogous to those for placing a pacemaker lead. Similarly, the controlling means that provides, for example, power and/or logic, can be placed within a patient's body using methods analogous to those for placing the pulse generator for a pacemaker.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An apparatus comprising an elongate support member having a first end and a second end, a controlling means positioned at said first end and an anchor means positioned at said second end, and two or more intracardiac devices positioned on or in said elongate support member between said first end and said second end, wherein said anchor means is adapted for attachment to the myocardium, and wherein said controlling means is adapted to control the action of said two or more intracardiac devices.

2. The apparatus of claim 1, wherein said two or more intracardiac devices are selected from the group consisting of valves, implantable cardioverter defibrillator (ICD) coils, balloons, micro pumps, and piezoelectric elements.

3. The apparatus of claim 1, wherein said two or more intracardiac devices comprise a first balloon and a second balloon, wherein said first balloon is adapted for placement at about the location of a cardiac valve, and wherein said second balloon is adapted for placement in the ventricle distal to said cardiac valve.

4. The apparatus of claim 3, wherein said cardiac valve is a mitral valve.

5. The apparatus of claim 1, wherein said two or more intracardiac devices comprise a valve and an ICD coil.

6. The apparatus of claim 5, wherein said ICD coil is distal to said valve.

7. The apparatus of claim 5, further comprising one or more electrodes on the circumference of said valve.

8. The apparatus of claim 1, wherein said two or more intracardiac devices comprise a valve and a balloon.

9. The apparatus of claim 8, wherein said balloon is distal to said valve and is adapted for placement within a ventricle.

10. The apparatus of claim 1, wherein said two or more cardiac devices comprise a mesh-like, conical-shaped valve.

11. The apparatus of claim 10, wherein said mesh-like, conical-shaped valve comprises one or more fabrics, polymers, pericardial tissue, fascial material, or a biological material coated with an anticoagulant.

12. The apparatus of claim 10, wherein said mesh-like, conical-shaped valve defines one or more openings adapted to permit unidirectional blood flow through said valve.

13. The apparatus of claim 1, comprising two or more elongate support members, a stent, and a valve, wherein said two or more elongate support members are attached to the outer surface of said stent.

14. The apparatus of claim 1, wherein said elongate support member is a hollow tube having a lumen extending between said first end and said second end, wherein said apparatus comprises a micro pump contained within said hollow tube, and wherein said hollow tube comprises a wall that defines one or more lateral openings adjacent to said micro pump such that said lumen is in fluid communication with the external surroundings of said elongate support member in the vicinity of said micro pump.

15. The apparatus of claim 1, wherein said two or more intracardiac devices comprise two or more piezoelectric elements.

16. The apparatus of claim 15, wherein each of said two or more piezoelectric elements comprise a radial array.

17. The apparatus of claim 15, wherein said two or more piezoelectric elements comprise at least one radial array and at least one linear array.

18. The apparatus of claim 15, wherein at least one of said piezoelectric elements is a radial array adapted for placement in the left atrium, at least one of said piezoelectric elements is a radial array adapted for placement at about the site of the mitral valve, and at least one of said piezoelectric elements is a radial array adapted for placement in the left ventricle.

19. The apparatus of claim 15, wherein at least one of said piezoelectric elements is a radial array adapted for placement in the right atrium, at least one of said piezoelectric elements is a radial array adapted for placement at about the site of the tricuspid valve, at least one of said piezoelectric elements is a radial array adapted for placement in the right ventricle, and at least one of said piezoelectric elements is a linear array adapted for placement in the right ventricle.

20. The apparatus of claim 1, wherein said anchor means is a screw, a hook, or a barb.

21. The apparatus of claim 1, wherein said apparatus is adapted for placement in the coronary sinus.
